(19) United States
(12) Patent Application Publication
(10) Pub. No.: US 2010/0104661 A1
(43) Pub. Date: Apr. 29, 2010

(54) DISCONTINUOUS METHODS OF TREATING CANCER

(76) Inventors: Susan Arbuck, Scotch Plains, NJ (US); Paul Kirschmeier, Basking Ridge, NJ (US); Brian Long, Clark, NJ (US); Genevieve A. Menard, Summit, NJ (US); Siu-Long Yao, West Windsor, NJ (US)

Correspondence Address: MERCK;PATENT DEPARTMENT (K-6-1, 1990), 2000 GALLOPING HILL ROAD, KENILWORTH, NJ, 07033-0530, US

(21) Appl. No.: 12/446,938

(22) PCT Filed: Oct. 23, 2007
(86) PCT No.: PCT/US07/22462

§ 371 Date: Dec. 15, 2009

Related U.S. Application Data
(60) Provisional application No. 60/854,343, filed on Oct. 25, 2006.
(60) Provisional application No. 60/860,206, filed on Nov. 20, 2006.

(51) Int. Cl. A61K 31/4545; A61K 33/24; A61P 35/00

(52) U.S. Cl. 424/649; 514/290

(57) ABSTRACT

Disclosed are methods of treating taxane sensitive tumors (i.e., cancers treatable with taxanes) using a taxane and the discontinuous dosing of lonafarnib.

### REFERENCE TO RELATED APPLICATION

This application claims the benefit of U.S. Provisional Application No. 60/854,342 filed Oct. 25, 2006, and U.S. Provisional Application No. 60/860,206 filed Nov. 20, 2006.

### BACKGROUND

Ovarian cancer remains the leading cause of gynecologic cancer deaths. Overall, ovarian cancer accounts for 4% of all cancer diagnosis in women and 5% of all cancer deaths. The majority of ovarian cancers (>90%) are epithelial in origin and often these cancers have already progressed to an advanced stage at the time of diagnosis.

In view of the interest in treating ovarian cancer, as well as other cancers (such as prostate cancer) a method of treating such cancers would be a welcome contribution to the art. This invention provides such a contribution.

### SUMMARY OF THE INVENTION

This invention provides a discontinuous dosing method of treating taxane sensitive tumors (i.e., cancers treatable with taxanes) in a patient in need of such treatment, said treatment comprising administering an effective amount of lonafarnib in combination with the standard of care therapy (e.g., the chemotherapeutics used) for said taxane sensitive tumors. Those skilled in the art will appreciate that the standard of therapy for taxane sensitive tumors comprises the administration of a taxane. The lonafarnib is administered in a discontinuous dosing schedule (e.g., lonafarnib is administered for only a part of a treatment cycle, e.g., lonafarnib is administered for a time period that is less than the total time period of the treatment cycle. In one example of the methods of this invention, lonafarnib is administered for up to seven days (e.g., 1 to about 7 days) of the treatment cycle. In another example of the methods of this invention, lonafarnib is administered for up to 5 days (e.g., 1 to about 5 days) of the treatment cycle. In another example of the methods of this invention, lonafarnib is administered for up to 3 days (e.g., 1 to about 3 days) of the treatment cycle.

Examples of cancers treatable in the methods of this invention include, for example, ovarian cancer (such epithelial ovarian cancer), prostate cancer, breast cancer, gastric cancer (stomach cancer), head and neck cancer, and lung cancer.

### BRIEF DESCRIPTION OF THE DRAWINGS

FIG. 1 shows the effect of intermittent dosing (3.5 days on/3.5 days off) with lonafarnib in combination with docetaxel (once weekly) on the growth of 22Rv1 prostate tumor xemografts.

FIG. 2 shows the results on day 21 from FIG. 1 wherein the results are represented as a bar graph.

FIG. 3 shows the effect of intermittent dosing (5 days on/5 days off) with lonafarnib in combination with docetaxel (once every 10 days) on the growth of 22Rv1 prostate tumor xemografts.

FIG. 4 shows the results on day 24 from FIG. 3 wherein the results are represented as a bar graph.

### DETAILED DESCRIPTION OF THE INVENTION

As used herein, unless otherwise defined, “patient” includes both human and animals. A “patient” is a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat. “Mammal” means humans and other mammalian animals.

As used herein, unless otherwise defined, “at least one” or “one or more” means there is 1 to several (e.g., 1, or 1 to 2, or 1 to 3 or 1 to 4, and the like).

As used herein, unless otherwise defined, “effective amount” or “therapeutically effective amount” is meant to describe an amount of drug, compound or composition effective in producing the desired therapeutic, ameliorative, inhibitory or preventative effect.

### Intermittent Dosing of Lonafarnib Around the Administration of Docetaxel in Prostate Cancer

Intermittent dosing of lonafarnib at 60 mpk BID around the administration of docetaxel was tested in two experiments using the 22Rv1 androgen-independent prostate cancer xenograft model. In both experiments 22Rv1 prostate tumor xenografts were grown in male athymic nude mice. When tumor volumes were approximately 100 mm³, the animals were randomized (n=10 per treatment group) for grouping. In both experiments the intermittent dosing of lonafarnib around the administration of docetaxel was superior to single-agent treatment with either agent alone. In addition, tumor regressions were observed when intermittent lonafarnib was combined with docetaxel.

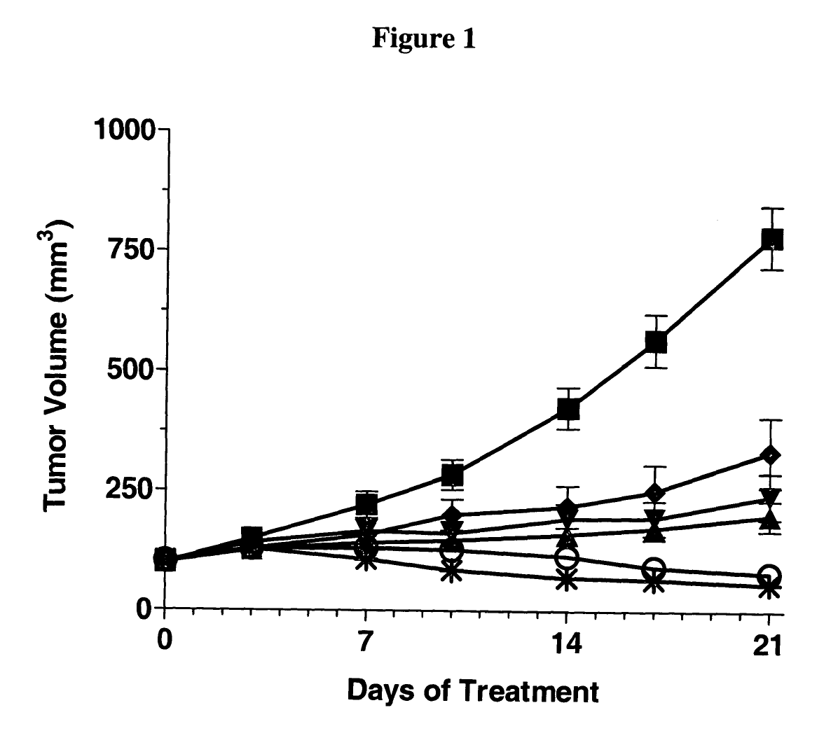

In the first experiment (see FIG. 1) male mice (n=10 per treatment group) bearing 22Rv1 tumor xenografts were treated with lonafarnib administered at either 30 mpk BID continuously or 60 mpk BID intermittently (3.5 days on/3.5 days off). Docetaxel was administered at 20 mpk once weekly after the fourth dose of lonafarnib. Tumor volumes were measured twice weekly. After 3 weeks (21 days) of treatment, single-agent docetaxel inhibited 22Rv1 tumor growth by 66%. Continuous treatment with single-agent lonafarnib at 30 mpk BID inhibited tumor growth by 85% and intermittent dosing of single-agent lonafarnib at 60 mpk BID inhibited tumor growth by 80%. When combined with weekly administration of docetaxel, continuous treatment with 30 mpk BID lonafarnib caused 22Rv1 tumors to regress by 23%. However, when combined with docetaxel, intermittent lonafarnib (60 mpk BID) caused the tumors to regress by 46%. Combination treatment with intermittent lonafarnib was significantly better than single-agent treatment with either agent alone (P<0.01).

FIG. 1 shows the effects of intermittent dosing (3.5 days on/3.5 days off) with lonafarnib in combination with docetaxel (once weekly) on the growth of 22Rv1 prostate tumor xenografts.

In FIG. 1:
▪ represents the tumor volumes (mean±standard error of the mean) in the animals treated with control vehicles.
♦ represents the tumor volumes (mean±standard error of the mean) in the animals treated with docetaxel (20 mpk, i.p., days 2, 9, 16).
▾ represents the tumor volumes (mean±standard error of the mean) in the animals treated with lonafarnib (60 mpk, p.o., BID, intermittent).

▴ represents the tumor volumes (mean±standard error of the mean) in the animals treated with lonafarnib (30 mpk, p.o., BID continuous).
◯ represents the tumor volumes (mean±standard error of the mean) in the animals treated with the combination of docetaxel (20 mpk, i.p., days 2, 9, 16) and lonafarnib (30 mpk, p.o., BID continuous).
represents the tumor volumes (mean±standard error of the mean) in the animals treated with the combination of docetaxel (20 mpk, i.p., days 2, 9, 16) and lonafarnib (60 mpk, p.o., BID, intermittent).

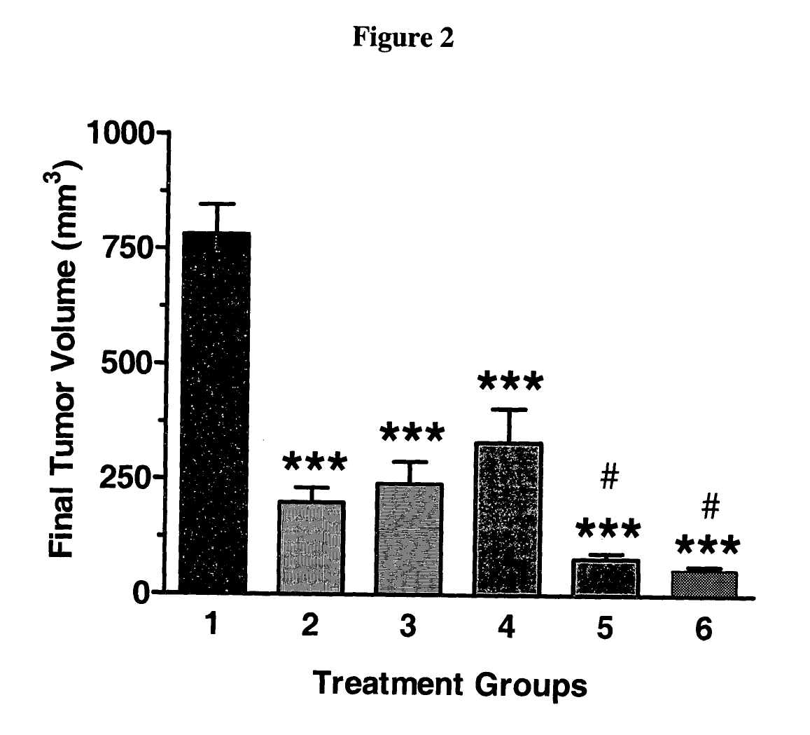

FIG. 2 shows the final volumes of the tumors after 21 days of treatment with intermittent (3.5 days on/3.5 days off) lonafarnib in combination with docetaxel (once weekly).

At the end of the experiment, final tumor volumes in the different treatment groups were measured. The bar graph (see FIG. 2) shows the final volumes per group of 10 animals for each of the different treatment groups.

In FIG. 2:
1 represents the tumor volumes (mean±standard error of the mean) in the animals treated with control vehicles.
2 represents the tumor volumes (mean±standard error of the mean) in the animals treated with lonafarnib (30 mpk, p.o., BID continuous).
3 represents the tumor volumes (mean±standard error of the mean) in the animals treated with lonafarnib (60 mpk, p.o., BID, intermittent).
4 represents the tumor volumes (mean±standard error of the mean) in the animals treated with docetaxel (20 mpk, i.p., days 2, 9, 16).
5 represents the tumor volumes (mean±standard error of the mean) in the animals treated with the combination of docetaxel (20 mpk, i.p., days 2, 9, 16) and lonafarnib (30 mpk, p.o., BID continuous).
6 represents the tumor volumes (mean±standard error of the mean) in the animals treated with the combination of docetaxel (20 mpk, i.p., days 2, 9, 16) and lonafarnib (60 mpk, p.o., BID, intermittent).
*** P<0.0001 versus vehicle-treated animals.
# P<0.01 versus single-agent lonafarnib and single-agent docetaxel treated animals.

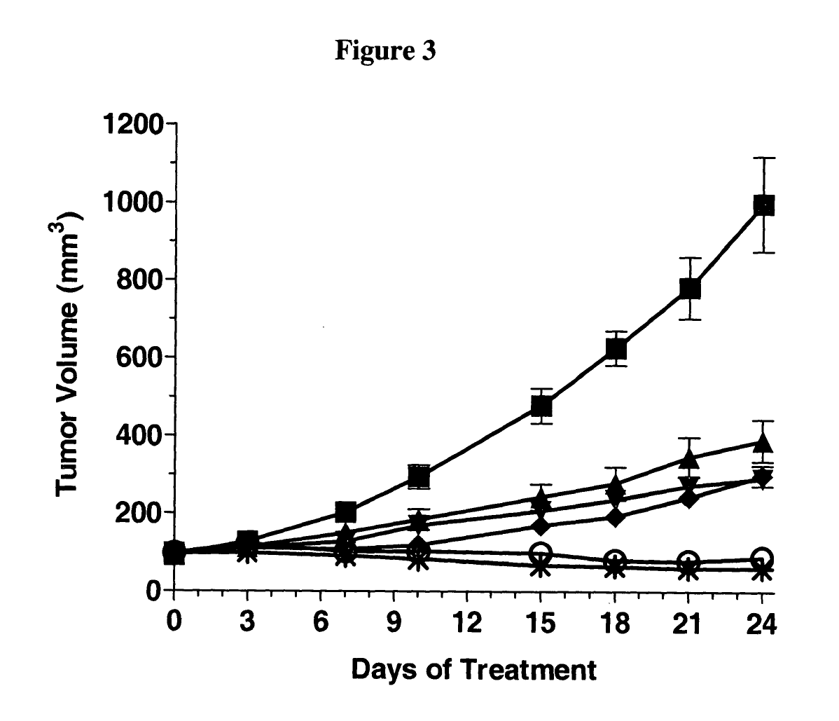

The second experiment (see FIG. 3) was also performed with male mice (n=10 per treatment group) bearing 22Rv1 prostate tumor xenografts that were approximately 100 mm³ in volume. Lonafarnib was administered at 30 mpk BID continuously or 60 mpk BID intermittently (5 days on/5 days off). Docetaxel was administered at 20 mpk once every 10 days after the second dose of lonafarnib. After 24 days of treatment, single-agent docetaxel inhibited 22Rv1 tumor growth by 78%. Continuous treatment with single-agent lonafarnib at 30 mpk BID inhibited tumor growth by 68% and intermittent dosing of single-agent lonafarnib at 60 mpk BID inhibited tumor growth by 78%. When combined with docetaxel, continuous treatment with 30 mpk BID lonafarnib caused 22Rv1 tumors to regress by 10%. However, when combined with docetaxel, intermittent lonafarnib (60 mpk BID) caused the tumors to regress by 37%. Combination treatment with intermittent lonafarnib was significantly better than single-agent treatment with either agent alone.

FIG. 3 shows the effects of intermittent dosing (5 days on/5 days off) with lonafarnib in combination with docetaxel (once every 10 days) on the growth of 22Rv1 prostate tumor xenografts.

In FIG. 3:
▪ represents the tumor volumes (mean±standard error of the mean) in the animals treated with control vehicles.
♦ represents the tumor volumes (mean±standard error of the mean) in the animals treated with docetaxel (20 mpk, i.p., days 1, 11, 21).
▾ represents the tumor volumes (mean±standard error of the mean) in the animals treated with lonafarnib (60 mpk, p.o., BID, intermittent).
▴ represents the tumor volumes (mean±standard error of the mean) in the animals treated with lonafarnib (30 mpk, p.o., BID continuous).
◯ represents the tumor volumes (mean±standard error of the mean) in the animals treated with the combination of docetaxel (20 mpk, i.p., days 1, 11, 21) and lonafarnib (30 mpk, p.o., BID continuous).
* represents the tumor volumes (mean±standard error of the mean) in the animals treated with the combination of docetaxel (20 mpk, i.p., days 2, 9, 16) and lonafarnib (60 mpk, p.o., BID, intermittent).

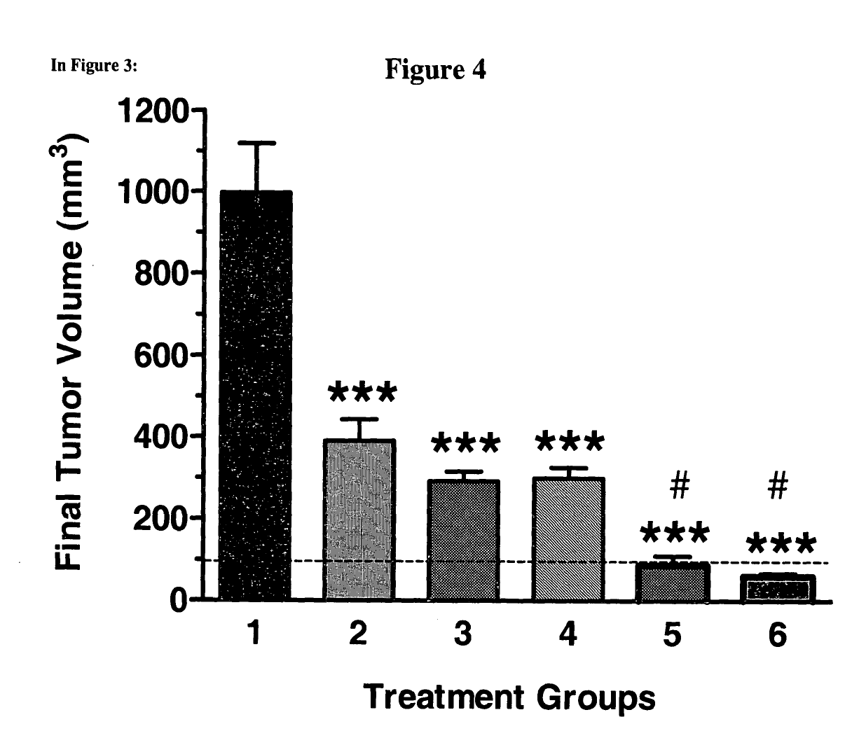

FIG. 4 shows the final volumes of the tumors after 24 days of treatment with intermittent (5 days on/5 days off) lonafarnib in combination with docetaxel (once every 10 days).

At the end of the experiment, final tumor volumes in the different treatment groups were measured. The bar graph (see FIG. 4) shows the final volumes per group of 10 animals for each of the different treatment groups.

In FIG. 4:
1 represents the tumor volumes (mean±standard error of the mean) in the animals treated with control vehicles.
2 represents the tumor volumes (mean±standard error of the mean) in the animals treated with lonafarnib (30 mpk, p.o., BID continuous).
3 represents the tumor volumes (mean±standard error of the mean) in the animals treated with lonafarnib (60 mpk, p.o., BID, intermittent).
4 represents the tumor volumes (mean±standard error of the mean) in the animals treated with docetaxel (20 mpk, i.p., days 1, 11, 21).
5 represents the tumor volumes (mean±standard error of the mean) in the animals treated with the combination of docetaxel (20 mpk, i.p., days 1, 11, 21) and lonafarnib (30 mpk, p.o., BID continuous).
6 represents the tumor volumes (mean±standard error of the mean) in the animals treated with the combination of docetaxel (20 mpk, i.p., days 1, 11, 21) and lonafarnib (60 mpk, p.o., BID, intermittent).
*** P<0.0001 versus vehicle-treated animals.
# P<0.01 versus single-agent lonafarnib and single-agent docetaxel treated animals.

The standard of care for particular cancers are well know to those skilled in the art. The standard of care is that treatment that experts agree is appropriate, accepted, and widely used. The standard of care is also called standard therapy or best practice. The chemotherapeutic agents used in the standard of care are used according to their know dosages and administration. See for example, the Physician's Desk Reference, 60^{th} edition, 2006, published by Thompson PDR at Montvale, N.J. 07645-1742, the disclosure of which is incorporated herein by reference thereto.

For example, Taxotere has the following label indications:
(1) Breast: Taxotere is indicated for the treatment of patients with locally advanced or metastatic breast cancer after failure of prior chemotherapy; and Taxotere in combo with doxorubicin and cyclophosphamide is indicated for the adjuvant treatment of patients with operable node-positive breast cancer;
(2) NSCLC: Taxotere as a single agent is indicated for the treatment of patients with locally advanced or metastatic non-small cell lung cancer after failure of prior platinum-based chemotherapy; and Taxotere in combination with cisplatin is indicated for the treatment of patients with unresectable, locally advanced or metastatic non-small cell lung cancer who have not previously received chemotherapy for this condition;
(3) Prostate: Taxotere in combination with prednisone is indicated for the treatment of patients with androgen independent (hormone refractory) metastatic prostate cancer;
(4) Gastric Adenocarcinoma: Taxotere in combination with cisplatin and fluorouracil is indicated for the treatment of patients with advanced gastric adenocarcinoma, including adenocarcinoma of the gastroesophageal junction, who have not received prior chemotherapy for advanced disease; and
(5) Head and Neck: Taxotere in combination with cisplatin and fluorouracil is indicated for the induction treatment of patients with inoperable locally advanced squamous cell carcinoma of the head and neck (SCCHN)

For example Taxol has the following label indications:
(1) Taxol is indicated as first-line and subsequent therapy for the treatment of advanced carcinoma of the ovary; As first-line therapy, Taxol is indicated in combination with cisplatin;
(2) Taxol is indicated for the adjuvant treatment of node-positive breast cancer administered sequentially to standard doxorubicin-containing combination chemotherapy; According to the label information, in the clinical trial, there was an overall favorable effect on disease-free and overall survival in the total population of patients with receptor-positive and receptor-negative tumors, but the benefit has been specifically demonstrated by available data (median follow-up 30 months) only in patients with estrogen and progesterone receptor negative tumors;
(3) Taxol is indicated for the treatment of breast cancer after failure of combination chemotherapy for metastatic disease or relapse within 6 months of adjuvant chemotherapy; Prior therapy should have included an anthracycline unless clinically contraindicated;
(4) Taxol in combination with cisplatin, is indicated for the first-line treatment of non-small cell lung cancer in patients who are not candidates for potentially curative surgery and/or radiation therapy; and
(5) Taxol is indicated for the second-line treatment of AIDS-related Kaposi's sarcoma.

Taxol can be administered in an amount of about 135 mg/m² to about 175 mg/m² as a 3-hour intravenous infusion.

For example, according to the labeling, for patients with carcinoma of the ovary, the following regimens are recommended:

(1) for previously untreated patients with carcinoma of the ovary, one of the following regimens may be given every three weeks (in selecting the appropriate regimen, differences in toxicities are considered by the skilled clinician):
(a) Taxol administered intravenously over 3 hours at a dose of 175 mg/m² followed by cisplatin at a dose of 75 mg/m²; or
(b) Taxol administered intravenously over 24 hours at a dose of 135 mg/m² followed by cisplatin at a dose of 75 mg/m²; and

(2) for patients previously treated with chemotherapy for carcinoma of the ovary, Taxol may be used at several doses and schedules; however, the optimal regimen is not yet clear; the recommended regimen is Taxol 135 mg/m² or 175 mg/m² administered intravenously over 3 hours every three weeks.

For patients with carcinoma of the breast, according to the labeling, the following regimens are recommended:

(1) for the adjuvant treatment of node-positive breast cancer, the recommended regimen is taxol, at a dose of 175 mg/m² intravenously over 3 hours every three weeks for four courses administered sequentially to doxorubicin-containing combination chemotherapy (e.g., cyclophosphamide at a dose of 600 mg/m² and doxorubicin at a doses of either 60 mg/m² (on day 1), 75 mg/m² (in two divided doses on days 1 and 2), or 90 mg/m² (in two divided doses on days 1 and 2 with prophylactic G-CSF support and ciprofloxacin)); and

(2) after failure of initial chemotherapy for metastatic disease or relapse within 6 months of adjuvant chemotherapy, Taxol at a dose of 175 mg/m² administered intravenously over 3 hours every three weeks.

For patients with non-small cell lung carcinoma, according to the labeling the recommended regimen, given every three weeks, is Taxol administered intravenously over 24 hours at a dose of 135 mg/m² followed by cisplatin, 75 mg/m².

For patients with AIDS-related Kaposi's sarcoma, according to the labeling, the recommended regimen is Taxol administered at a dose of 135 mg/m² given intravenously over 3 hours every three weeks or at a dose of 100 mg/m² given intravenously over 3 hours every 2 weeks (dose intensity 45-50 mg/m²/week). According to the labeling, in the two clinical trials evaluating these schedules, the former schedule (135 mg/m² every 3 weeks) was more toxic than the latter. In addition, according to the labeling, all patients with low performance status were treated with the latter schedule (100 mg/m² every 2 weeks). According to the labeling, based upon the immunosuppression in patients with advanced HIV disease, the following modifications are recommended in these patients: (1) reduce the dose of dexamethasone as one of the three premedication drugs to 10 mg PO (instead of 20 mg PO); (2) initiate or repeat treatment with Taxol only if the neutrophil count is at least 1000 cells/mm³; (3) reduce the dose of subsequent courses of Taxol by 20% for patients who experience severe neutropenia (neutrophil less than 500 cells/mm³ for a week or longer); and (4) initiate concomitant hematopoietic growth factor (G-CSF) as clinically indicated.

According to the labeling, for therapy of patients with solid tumors (ovary, breast, NSCLC (non small cell lung cancer)), courses of Taxol should not be repeated until the neutrophil count is at least 1500 cells/mm³ and the platelet count is at least 100,000 cells/mm³. Taxol should not be given to patients with AIDs-related Kaposi's sarcoma if the baseline or subsequent neutrophil count is less than 1000 cells/mm³. Patients who experience severe neutropenia (neutrophil less than 500 cells/mm³ for a week or longer) or severe peripheral neuropathy during Taxol therapy should have dosages reduced by 20% for subsequent courses of Taxol. The incidence of neurotoxicity and the severity of neutropenia increase with dose.

Taxotere is used to treat breast cancer, NSCLC, prostate cancer, stomach cancer, and head and neck cancer.

According to the labeling, for the treatment of breast cancer, the recommended dosage of Taxotere is 60-100 mg/m² administered intravenously over 1 hour every 3 weeks. In the adjuvant treatment of operable node-positive breast cancer, the recommended Taxotere dose is 75 mg/m² administered 1-hour after doxorubicin 50 mg/m² and cyclophosphamide 500 mg/m² every 3 weeks for 6 courses. Prophylactic G-CSF may be used to mitigate the risk of hematological toxicities.

According to the labeling, for treatment of NSCLC after failure of prior platinum-based chemotherapy, Taxotere was evaluated as monotherapy, and the recommended dose is 75 mg/m² administered intravenously over 1 hour every three weeks. According to the labeling, a dose of 100 mg/m² in patients previously treated with chemotherapy was associated with increased hematologic toxicity, infection, and treatment related mortality in randomized, controlled trials. According to the labeling, for chemotherapy-naïve patients, Taxotere was evaluated in combination with cisplatin. The recommended dose of Taxotere is 75 mg/m² administered over 1 hour immediately followed by cisplatin 75 mg/m² over 30-60 minutes every 3 weeks.

According to the labeling, for the treatment of hormone-refractory metastatic prostate cancer, the recommended dose of Taxotere is 75 mg/m² every 3 weeks as a 1 hour intravenous infusion. Prednisone 5 mg orally twice daily is administered continuously.

According to the labeling, for gastric adenocarcinoma, the recommended dose of Taxotere is 75 mg/m² as a 1 hour intravenous infusion, followed by cisplatin 75 mg/m², as a 1 to 3 hour intravenous infusion (both on day 1 only), followed by fluorouracil 750 mg/m² per day given as a 24 hour continuous intravenous infusion for 5 days, starting at the end of the cisplatin infusion. According to the labeling, treatment is repeated every three weeks. (Patients receive premedication with antiemetics and appropriate hydration for cisplatin administration.

For the treatment of head and neck cancer, according to the labeling, for the induction treatment of locally advanced inoperable SCCHN, the recommended dose of Taxotere is 75 mg/m² as a 1 hour intravenous infusion followed by cisplatin 75 mg/m² intravenously over 1 hour, on day one, followed by fluorouracil as a continuous intravenous infusion at 750 mg/m² per day for five days. According to the labeling, this regimen is administered every 3 weeks for 4 cycles. According to the labeling, following chemotherapy, patients should receive radiotherapy. According to the labeling, Patients must receive premedication with antiemetics and appropriate hydration (prior to and after cisplatin administration).

According to the Taxotere labeling, all patients should be premedicated with oral corticosteroids such as dexamethasone 16 mg per day (e.g., 8 mg BID) for 3 days starting 1 day prior to taxotere administration in order to reduce the incidence and severity of fluid retention as well as the severity of hypersensitivity reactions. According to the labeling, for hormone-refractory metastatic prostate cancer, given the concurrent use of prednisone, the recommended premedication regimen is oral dexamethasone 8 mg, at 12 hours, 3 hours and 1 hour before Taxotere infusion.

As the skilled clinician will appreciate, the doses of Taxotere can be adjusted during treatment in response to observed drug reactions. Such recommended dosage adjustments are described in the labeling for Taxotere (see for example, the PDR cited above and incorporated by reference).

Thus, in one embodiment this invention provides a discontinuous dosing method of treating taxane sensitive tumors (i.e., cancers treatable with taxanes) in a patient in need of such treatment, said treatment comprising the administration of an effective amount of lonafarnib in combination with an effective amount of a taxane, and optionally, an effective amount a platinum coordinator complex. The lonafarnib is administered in a discontinuous dosing schedule (e.g., lonafarnib is administered for only a part of a treatment cycle, e.g., lonafarnib is administered for a time period that is less than the total time period of the treatment cycle. In one example of the methods of this invention, lonafarnib is administered for up to seven days (e.g., 1 to about 7 days) of the treatment cycle. In another example of the methods of this invention, lonafarnib is administered for up to 5 days (e.g., 1 to about 5 days) of the treatment cycle. In another example of the methods of this invention, lonafarnib is administered for up to 3 days (e.g., 1 to about 3 days) of the treatment cycle.

In another embodiment this invention provides a method of treating cancer in a patient in need of such treatment, said treatment comprising administering lonafarnib (e.g., the Sarasar® brand of lonafarnib) in combination with an effective amount of: (1) a taxane (such as, for example, paclitaxel or docetaxel), and (2) optionally, a platinum coordinator complex (such as, for example, carboplatin, cisplatin or oxaliplatin), wherein said lonafarnib is administered in a discontinuous dosing schedule.

In another embodiment this invention provides a method of treating cancer in a patient in need of such treatment, said treatment comprising administering an effective amount of lonafarnib (e.g., the Sarasar® brand of lonafarnib) in combination with an effective amount of at least one chemotherapeutic agent selected from the group consisting of: (1) a taxane selected from the group consisting of: paclitaxel (e.g., the Taxol® brand of paclitaxel, or the Abraxane® brand of paclitaxel) and docetaxel (e.g., the Taxotere® brand of docetaxel), and (2) a platinum coordinator complex selected from the group consisting of: carboplatin, cisplatin and oxaliplatin (e.g., the Eloxatin® brand of oxaliplatin), wherein said lonafarnib is administered in a discontinuous dosing schedule.

This invention also provides a method of treating cancer) in a patient in need of such treatment, said treatment comprising administering an effective amount of lonafarnib (e.g., the Sarasar® brand of lonafarnib) in combination with an effective amount of a taxane selected from the group consisting of: paclitaxel (e.g., the Taxol® brand of paclitaxel, or the Abraxane® brand of paclitaxel) and docetaxel (e.g., the Taxotere® brand of docetaxel), wherein said lonafarnib is administered in a discontinuous dosing schedule.

Thus, in the methods of this invention, lonafarnib is administered for only a part of the treatment cycle, i.e., lonafarnib is administered for a time period that is less than the total time period of the treatment cycle. For example, lonafarnib is administered for up to 7 days (e.g., 1 to about 7 days). In another example, lonafarnib is administered for up to 5 days (e.g., 1 to about 5 days). In another example, lonafarnib is administered for up to 3 days (e.g., 1 to about 3 days). Usually the administration of lonafarnib starts on the first day of the treatment cycle. Lonafarnib can be administered starting the day before the treatment cycle starts (i.e., the day before the first day of the treatment cycle, i.e., day 0). Thus, for example, in a 21 day treatment cycle, lonafarnib is administered for less than 21 days. For example, in a 21 day treatment cycle, lonafarnib is administered for up to 7 days (e.g., 1 to about 7 days). In another example, in a 21 day treatment cycle, lonafarnib is administered for up to 5 days (e.g., 1 to about 5 days). In another example, in a 21 day treatment cycle, lonafarnib is administered for up to 3 days (e.g., 1 to about 3 days). In another example, in a 21 day treatment cycle, lonafarnib is administered for up to 7 days (e.g., 1 to about 7 days) starting on day 1 of the treatment cycle. In another example, in a 21 day treatment cycle, lonafarnib is administered for up to 5 days (e.g., 1 to about 5 days) starting on day 1 of the treatment cycle. In another example, in a 21 day treatment cycle, lonafarnib is administered for up to 3 days (e.g., 1 to about 3 days) starting on day 1 of the treatment cycle.

Usually the administration of lonafarnib is continuous for the number of days administered in a treatment cycle. Thus, when lonafarnib is administered, for example, for 1 to 7 days of the treatment cycle, the administration is continuous from the first day of administration to the seventh day of administration. Similarly, when lonafarnib is administered, for example, for 1 to 5 days of the treatment cycle, the administration is continuous from the first day of administration to the fifth day of administration. And similarly, when lonafarnib is administered, for example, for 1 to 3 days of the treatment cycle, the administration is continuous from the first day of administration to the fifth day of administration.

In one embodiment of this invention, the lonafarnib is administered starting on day 0 of the treatment cycle, and the chemotherapeutic agent or agents are administered on the first day of the treatment cycle.

In one embodiment of this invention, the lonafarnib is administered starting on day 1 of the treatment cycle, and the chemotherapeutic agent or agents are administered on the second day of the treatment cycle.

In another embodiment of this invention, the lonafarnib is administered starting on day 1 of the treatment cycle, and the chemotherapeutic agent or agents are on day 1 of the treatment cycle.

Generally, in the methods of this invention, lonafarnib is administered in an amount of about 25 to about 200 mg PO BID (i.e., orally twice per day) for each day of administration during the treatment cycle. In another example, lonafarnib is administered in an amount of about 100 mg PO BID. In another example, lonafarnib is administered in an amount of about 150 to about 200 mg PO BID. In one example, lonafarnib is administered in an amount of about 200 mg PO BID. Usually, the doses of lonafarnib are given 12 hours apart, and usually the doses of lonafarnib are given 12 hours apart with food.

Generally, paclitaxel is administered in an amount of about 135 mg/m² to about 185 mg/m² once during the treatment cycle.

Usually paclitaxel is given once on day 1 of each treatment cycle. Usually paclitaxel is given as an infusion, and usually as a about a 3 hour infusion. Usually paclitaxel is administered in an amount of about 135 mg/m² to about 185 mg/m² once during the treatment cycle. Paclitaxel is generally administered at a dose of about 175 mg/m². Thus, paclitaxel is generally administered as about a 3 hour infusion at a dose of 175 mg/m².

Generally carboplatin is administered in an amount to provide an AUC (mg/ml×min) of about 4 to about 6 once during the treatment cycle.

Usually, carboplatin, is given once on day 1 of each treatment cycle. Usually carboplatin is administered as an infusion, and usually as about a 30 minute infusion. Usually, carboplatin is administered in an amount to provide an AUC (mg/ml×min) of about 4 to about 6. Carboplatin is generally administered in an amount to provide an AUC of about 5 (mg/ml×min). Thus, carboplatin is generally administered as a about a 30 minute infusion at a dose to provide and AUC of about 5 (mg/ml×min).

When carboplatin and paclitaxel are administered in the method of treatment, generally the infusion of paclitaxel is given first followed by the infusion of carboplatin.

Generally cisplatin is administered once during the treatment cycle in an amount of about 30 mg/m² to about 100 mg/m² (for example, 75 mg/m²).

Generally, oxaliplatin (e.g., the Eloxatin® brand of oxaliplatin) is administered once during the treatment cycle in an amount of 50-100 mg/m².

Generally, docetaxel (e.g., the Taxotere® brand of docetaxel) is administered once during the treatment cycle in an amount of about 50 to about 100 mg/m² (for example, 75 mg/m²).

The starting doses of the therapeutic agents (e.g., the lonafarnib, the taxane and the platinum coordinator complex, and usually the taxane and the platinum coordinator complex, or either the taxane or platinum coordinator complex) can be adjusted by the skilled clinician in response to toxicity side effects in the patient.

Usually, in the embodiments of this invention, more than one treatment cycle is administered, i.e., usually the treatment cycle is repeated. Thus, in one embodiment of this invention the treatment cycle is repeated up to six times. In another embodiment of this invention the treatment cycle is repeated six times.

Generally, a treatment cycle is 21 days.

Generally, multiple treatment cycles are administered, i.e., the treatment cycle is repeated. For example, the treatment cycle can be repeated for up to 6 times.

At the end of the treatment cycles lonafarnib can be administered as a monotherapy. In the monotherapy Lonafarnib can be administered in an amount of about 25 to about 200 mg PO BID. Usually, lonafarnib is administered at a dose of 200 mg PO BID. Usually each dose is administered 12 hours apart, and usually each dose is administered with food. The monotherapy with lonafarnib can be continued as long as the patient experiences stable disease, NED (no evidence of disease) or objective response (CR/PR), and manageable toxicity. Thus, for example, the lonafarnib monotherapy can be continued for up to 6 months (e.g., 6 months) after cessation of the combination therapy (i.e., after cessation of the therapy with the taxane and the platinum coordinator complex).

One embodiment of this invention is directed to treating ovarian cancer. Epithelial ovarian cancer is an example of the ovarian cancer treated in the methods of this invention.

Another embodiment of this invention is directed to treating prostate cancer.

Another embodiment of this invention is directed to treating breast cancer.

Another embodiment is directed to the treatment of lung cancer.

Another embodiment is directed to the treatment of gastric cancer.

Another embodiment of this invention is directed to the treatment of cancer of the head and neck.

Generally, in the treatment of ovarian cancer (e.g., epithelial ovarian cancer) carboplatin and paclitaxel are used.

One embodiment of this invention is directed to a method of treating ovarian cancer (e.g., epithelial ovarian cancer) in a patient in need of such treatment, said treatment comprising administering lonafarnib in combination with paclitaxel (e.g., the Taxol® brand of paclitaxel, or the Abraxane® brand of paclitaxel), and carboplatin, wherein said lonafarnib is administered in a discontinuous dosing schedule.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to about 7 days of the treatment cycle in an amount of about 25 to about 200 mg PO BID (i.e., orally twice per day) for each day of administration in the treatment cycle;

(b) said paclitaxel is administered in an amount of about 135 mg/m² to about 185 mg/m² once during the treatment cycle; and

(c) said carboplatin is administered in an amount to provide an AUC (mg/ml×min) of about 4 to about 6 once during the treatment cycle.

Generally, in the treatment of ovarian cancer the treatment cycle is 21 days.

Thus, another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 7 days in an amount of about 100 mg twice per day on each day of administration;

(b) said paclitaxel is administered in an amount of about 175 mg/m² once during the treatment cycle; and

(c) said carboplatin is administered in an amount to provide an AUC (mg/ml×min) of about 5 once during the treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 7 days in an amount of about 100 mg twice per day on each day of administration;

(b) said paclitaxel is administered in an amount of about 175 mg/m² once on day 1 of each treatment cycle; and

(c) said carboplatin is administered in an amount to provide an AUC (mg/ml×min) of about 5 once on day 1 of each treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 7 days in an amount of about 100 mg twice per day on each day of administration, wherein each dose is administered about 12 hours apart from the previous dose;

(b) said paclitaxel is administered as about a 3 hour infusion in an amount of about 175 mg/m² once on day 1 of each treatment cycle; and

(c) said carboplatin is administered as a 30 minute infusion in an amount to provide an AUC (mg/ml×min) of about 5 once on day 1 of each treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 7 days in an amount of about 100 mg twice per day on each day of administration, wherein each dose is administered about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle; and

(d) said carboplatin is administered after said paclitaxel.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 7 days in an amount of about 100 mg twice per day on each day of administration, wherein each dose is administered with food about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle; and

(d) said carboplatin is administered after said paclitaxel.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 7 days in an amount of about 100 mg twice per day on each day of administration, wherein each dose is administered with food about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle;

(d) said carboplatin is administered after said paclitaxel; and

(e) after the last treatment cycle (i.e., after cessation of combination therapy with paclitaxel and carboplatin), lonafarnib is administered continuously as a monotherapy at a dose of about 200 mg PO BID (usually each dose being administered 12 hours apart, and usually each dose being administered with food), and usually the monotherapy is continued for up to 6 months.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to about 5 days of the treatment cycle in an amount of about 25 to about 200 mg PO BID (i.e., orally twice per day) for each day of administration in the treatment cycle;

(b) said paclitaxel is administered in an amount of about 135 mg/m² to about 185 mg/m² once during the treatment cycle; and

(c) said carboplatin is administered in an amount to provide an AUC (mg/ml×min) of about 4 to about 6 once during the treatment cycle.

Thus, another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 100 mg twice per day on each day of administration;

(b) said paclitaxel is administered in an amount of about 175 mg/m² once during the treatment cycle; and

(c) said carboplatin is administered in an amount to provide an AUC (mg/ml×min) of about 5 once during the treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 100 mg twice per day on each day of administration;

(b) said paclitaxel is administered in an amount of about 175 mg/m² once on day 1 of each treatment cycle; and

(c) said carboplatin is administered in an amount to provide an AUC (mg/ml×min) of about 5 once on day 1 of each treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 100 mg twice per day on each day of administration, wherein each dose is administered about 12 hours apart from the previous dose;

(b) said paclitaxel is administered as about a 3 hour infusion in an amount of about 175 mg/m² once on day 1 of each treatment cycle; and

(c) said carboplatin is administered as a 30 minute infusion in an amount to provide an AUC (mg/ml×min) of about 5 once on day 1 of each treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 100 mg twice per day on each day of administration, wherein each dose is administered about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle; and

(d) said carboplatin is administered after said paclitaxel.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 100 mg twice per day on each day of administration, wherein each dose is administered with food about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle; and

(d) said carboplatin is administered after said paclitaxel.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 100 mg twice per day on each day of administration, wherein each dose is administered with food about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle;

(d) said carboplatin is administered after said paclitaxel; and

(e) after the last treatment cycle (i.e., after cessation of combination therapy with paclitaxel and carboplatin), lonafarnib is administered continuously as a monotherapy at a dose of about 200 mg PO BID (usually each dose being administered 12 hours apart, and usually each dose being administered with food), and usually the monotherapy is continued for up to 6 months.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 7 days in an amount of about 200 mg twice per day on each day of administration;

(b) said paclitaxel is administered in an amount of about 175 mg/m² once during the treatment cycle; and

(c) said carboplatin is administered in an amount to provide an AUC (mg/ml×min) of about 5 once during the treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 7 days in an amount of about 200 mg twice per day on each day of administration;

(b) said paclitaxel is administered in an amount of about 175 mg/m² once on day 1 of each treatment cycle; and

(c) said carboplatin is administered in an amount to provide an AUC (mg/ml×min) of about 5 once on day 1 of each treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 7 days in an amount of about 200 mg twice per day on each day of administration, wherein each dose is administered about 12 hours apart from the previous dose;

(b) said paclitaxel is administered as about a 3 hour infusion in an amount of about 175 mg/m² once on day 1 of each treatment cycle; and

(c) said carboplatin is administered as a 30 minute infusion in an amount to provide an AUC (mg/ml×min) of about 5 once on day 1 of each treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 7 days in an amount of about 200 mg twice per day on each day of administration, wherein each dose is administered about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle; and

(d) said carboplatin is administered after said paclitaxel.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 7 days in an amount of about 200 mg twice per day on each day of administration, wherein each dose is administered with food about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle; and

(d) said carboplatin is administered after said paclitaxel.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 7 days in an amount of about 200 mg twice per day on each day of administration, wherein each dose is administered with food about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle;

(d) said carboplatin is administered after said paclitaxel; and

(e) after the last treatment cycle (i.e., after cessation of combination therapy with paclitaxel and carboplatin), lonafarnib is administered continuously as a monotherapy at a dose of about 200 mg PO BID (usually each dose being administered 12 hours apart, and usually each dose being administered with food), and usually the monotherapy is continued for up to 6 months.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to about 5 days of the treatment cycle in an amount of about 25 to about 200 mg PO BID (i.e., orally twice per day) for each day of administration in the treatment cycle;

(b) said paclitaxel is administered in an amount of about 135 mg/m² to about 185 mg/m² once during the treatment cycle; and

(c) said carboplatin is administered in an amount to provide an AUC (mg/ml×min) of about 4 to about 6 once during the treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 100 mg twice per day on each day of administration;

(b) said paclitaxel is administered in an amount of about 175 mg/m² once during the treatment cycle; and

(c) said carboplatin is administered in an amount to provide an AUC (mg/ml×min) of about 5 once during the treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 100 mg twice per day on each day of administration;

(b) said paclitaxel is administered in an amount of about 175 mg/m² once on day 1 of each treatment cycle; and

(c) said carboplatin is administered in an amount to provide an AUC (mg/ml×min) of about 5 once on day 1 of each treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 100 mg twice per day on each day of administration, wherein each dose is administered about 12 hours apart from the previous dose;

(b) said paclitaxel is administered as about a 3 hour infusion in an amount of about 175 mg/m² once on day 1 of each treatment cycle; and

(c) said carboplatin is administered as a 30 minute infusion in an amount to provide an AUC (mg/ml×min) of about 5 once on day 1 of each treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 100 mg twice per day on each day of administration, wherein each dose is administered about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle; and

(d) said carboplatin is administered after said paclitaxel.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 100 mg twice per day on each day of administration, wherein each dose is administered with food about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle; and

(d) said carboplatin is administered after said paclitaxel.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 100 mg twice per day on each day of administration, wherein each dose is administered with food about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle;

(d) said carboplatin is administered after said paclitaxel; and

(e) after the last treatment cycle (i.e., after cessation of combination therapy with paclitaxel and carboplatin), lonafarnib is administered continuously as a monotherapy at a dose of about 200 mg PO BID (usually each dose being administered 12 hours apart, and usually each dose being administered with food), and usually the monotherapy is continued for up to 6 months.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 200 mg twice per day on each day of administration;

(b) said paclitaxel is administered in an amount of about 175 mg/m² once during the treatment cycle; and

(c) said carboplatin is administered in an amount to provide an AUC (mg/ml×min) of about 5 once during the treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 200 mg twice per day on each day of administration;

(b) said paclitaxel is administered in an amount of about 175 mg/m² once on day 1 of each treatment cycle; and

(c) said carboplatin is administered in an amount to provide an AUC (mg/ml×min) of about 5 once on day 1 of each treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 200 mg twice per day on each day of administration, wherein each dose is administered about 12 hours apart from the previous dose;

(b) said paclitaxel is administered as about a 3 hour infusion in an amount of about 175 mg/m² once on day 1 of each treatment cycle; and

(c) said carboplatin is administered as a 30 minute infusion in an amount to provide an AUC (mg/ml×min) of about 5 once on day 1 of each treatment cycle.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 200 mg twice per day on each day of administration, wherein each dose is administered about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle; and

(d) said carboplatin is administered after said paclitaxel.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 200 mg twice per day on each day of administration, wherein each dose is administered with food about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle; and

(d) said carboplatin is administered after said paclitaxel.

Another embodiment of this invention is directed to a method of treating ovarian cancer in a patient in need of such treatment, said treatment comprising administering lonafarnib in combination with paclitaxel and carboplatin, wherein:

(a) said lonafarnib is administered continuously for 1 to 5 days in an amount of about 200 mg twice per day on each day of administration, wherein each dose is administered with food about 12 hours apart from the previous dose;

(b) said paclitaxel is administered as about a 3 hour infusion in an amount of about 175 mg/m² once on day 1 of each treatment cycle;

(c) said carboplatin is administered as a 30 minute infusion in an amount to provide an AUC (mg/ml×min) of about 5 once on day 1 of each treatment cycle;

(d) said carboplatin is administered after said paclitaxel; and

(e) after the last treatment cycle (i.e., after cessation of combination therapy with paclitaxel and carboplatin), lonafarnib is administered continuously as a monotherapy at a dose of about 200 mg PO BID (usually each dose being administered 12 hours apart, and usually each dose being administered with food), and usually the monotherapy is continued for up to 6 months.

Another embodiment of this invention is directed to any one of the methods of treating ovarian cancer described herein wherein the administration of lonafarnib starts on day 1 of the treatment cycle.

Another embodiment of this invention is directed to any one of the methods of treating ovarian cancer wherein a treatment cycle is for 21 days.

Another embodiment of this invention is directed to any one of the methods of treating ovarian cancer wherein up to six treatment cycles are administered.

Another embodiment of this invention is directed to any one of the methods of treating ovarian cancer wherein six treatment cycles are administered.

Carboplatin is the preferred platinum coordinator complex used in the methods of treating ovarian cancer. Other platinum coordinator complexes, such as, for example, cisplatin or oxaliplatin, can be used in place of carboplatin. For example, the chemotherapeutic agent cisplatin can be used in an amount of about 30 mg/m² to about 100 mg/m² (for example, 75 mg/m²). Also, for example, the chemotherapeutic agent oxaliplatin (Eloxatin® brand of oxaliplatin) can be administered in an amount of 50-100 mg/m².

Paclitaxel is the preferred taxane used in the methods of treating ovarian cancer. Other taxanes, such as, for example, docetaxel, can be used in place of paclitaxel. For example, docetaxel (e.g., the Taxotere® brand of docetaxel) can be administered in an amount of about 50 to about 100 mg/m² (for example, 75 mg/m²).

Another embodiment of this invention is directed to a method of treating prostate cancer in a patient in need of such treatment, said treatment comprising administering an effective amount of lonafarnib (e.g., the Sarasar® brand of lonafarnib) in combination with an effective amount of a taxane (such as, for example, paclitaxel or docetaxel), wherein said lonafarnib is administered in a discontinuous dosing schedule.

Another embodiment of this invention is directed to a method of treating prostate cancer in a patient in need of such treatment, said treatment comprising administering an effective amount of lonafarnib in combination with an effective amount of docetaxel, wherein said lonafarnib is administered in a discontinuous dosing schedule. In one embodiment said lonafarnib is administered continuously for 1 to about 7 days in the treatment cycle. In another embodiment said lonafarnib is administered continuously for 1 to about 5 days in the treatment cycle. Generally, each treatment cycle is about 21 days. Generally, each treatment cycle can be repeated. For example, each treatment cycle can be repeated for up to six times. In one example the treatment cycle can be repeated for six times. After the last treatment cycle lonafarnib can be continued as a monotherapy. In the monotherapy lonafarnib can be administered in an amount of about 25 to about 200 mg PO BID. Usually, lonafarnib is administered at a dose of 200 mg PO BID. Usually each dose is administered 12 hours apart, and usually each dose is administered with food. The monotherapy with lonafarnib can be continued as long as the patient experiences stable disease, NED (no evidence of disease) or objective response (CR/PR), and manageable toxicity. Thus, for example, the lonafarnib monotherapy can be continued for 6 months after cessation of the combination therapy (i.e., after cessation of the therapy with docetaxel).

Another embodiment of this invention is directed to a method of treating prostate cancer in a patient in need of such treatment, said treatment comprising administering an effective amount of lonafarnib in combination with an effective amount of docetaxel wherein:

(a) said lonafarnib is administered continuously for 1 to about 7 days of the treatment cycle in an amount of about 25 to about 200 mg PO BID (i.e., orally twice per day) for each day of administration in the treatment cycle; and

(b) said docetaxel is administered in an amount of about 50 to about 100 mg/m² (for example, 75 mg/m²) once in the treatment cycle.

Another embodiment of this invention is directed to a method of treating prostate cancer in a patient in need of such treatment, said treatment comprising administering an effective amount of lonafarnib in combination with an effective amount of docetaxel wherein:

(a) said lonafarnib is administered continuously for 1 to about 7 days of the treatment cycle in an amount of about 25 to about 200 mg PO BID (i.e., orally twice per day) for each day of administration in the treatment cycle, wherein each dose is administered about 12 hours apart from the previous dose; and

(b) said docetaxel is administered in an amount of about 50 to about 100 mg/m² (for example, 75 mg/m²) once in the treatment cycle.

Another embodiment of this invention is directed to a method of treating prostate cancer in a patient in need of such treatment, said treatment comprising administering an effective amount of lonafarnib in combination with an effective amount of docetaxel wherein:

(a) said lonafarnib is administered continuously for 1 to about 7 days of the treatment cycle in an amount of about 25 to about 200 mg PO BID (i.e., orally twice per day) for each day of administration in the treatment cycle, wherein each dose is administered about 12 hours apart from the previous dose, and wherein each does is administered with food; and

(b) said docetaxel is administered in an amount of about 50 to about 100 mg/m² (for example, 75 mg/m²) once in the treatment cycle.

Another embodiment of this invention is directed to a method of treating prostate cancer in a patient in need of such treatment, said treatment comprising administering an effective amount of lonafarnib in combination with an effective amount of docetaxel wherein:

(a) said lonafarnib is administered continuously for 1 to about 7 days of the treatment cycle in an amount of about 200 mg PO BID (i.e., orally twice per day) for each day of administration in the treatment cycle; and

(b) said docetaxel is administered in an amount of about 75 mg/m² once in the treatment cycle.

Another embodiment of this invention is directed to a method of treating prostate cancer in a patient in need of such treatment, said treatment comprising administering an effective amount of lonafarnib in combination with an effective amount of docetaxel wherein:

(a) said lonafarnib is administered continuously for 1 to about 7 days of the treatment cycle in an amount of about 200 mg PO BID (i.e., orally twice per day) for each day of administration in the treatment cycle, wherein each dose is administered about 12 hours apart from the previous dose; and

(b) said docetaxel is administered in an amount of about, 75 mg/m² once in the treatment cycle.

Another embodiment of this invention is directed to a method of treating prostate cancer in a patient in need of such treatment, said treatment comprising administering an effective amount of lonafarnib in combination with an effective amount of docetaxel wherein:

(a) said lonafarnib is administered continuously for 1 to about 7 days of the treatment cycle in an amount of about 200 mg PO BID (i.e., orally twice per day) for each day of administration in the treatment cycle, wherein each dose is administered about 12 hours apart from the previous dose, and wherein each does is administered with food; and

(b) said docetaxel is administered in an amount of about 75 mg/m² once in the treatment cycle.

Another embodiment of this invention is directed to a method of treating prostate cancer in a patient in need of such treatment, said treatment comprising administering an effective amount of lonafarnib in combination with an effective amount of docetaxel wherein:

(a) said lonafarnib is administered continuously for 1 to about 5 days of the treatment cycle in an amount of about 25 to about 200 mg PO BID (i.e., orally twice per day) for each day of administration in the treatment cycle; and

(b) said docetaxel is administered in an amount of about 50 to about 100 mg/m² (for example, 75 mg/m²) once in the treatment cycle.

Another embodiment of this invention is directed to a method of treating prostate cancer in a patient in need of such treatment, said treatment comprising administering an effective amount of lonafarnib in combination with an effective amount of docetaxel wherein:

(a) said lonafarnib is administered continuously for 1 to about 5 days of the treatment cycle in an amount of about 25 to about 200 mg PO BID (i.e., orally twice per day) for each day of administration in the treatment cycle, wherein each dose is administered about 12 hours apart from the previous dose; and

(b) said docetaxel is administered in an amount of about 50 to about 100 mg/m² (for example, 75 mg/m²) once in the treatment cycle.

Another embodiment of this invention is directed to a method of treating prostate cancer in a patient in need of such treatment, said treatment comprising administering an effective amount of lonafarnib in combination with an effective amount of docetaxel wherein:

(a) said lonafarnib is administered continuously for 1 to about 5 days of the treatment cycle in an amount of about 25 to about 200 mg PO BID (i.e., orally twice per day) for each day of administration in the treatment cycle, wherein each dose is administered about 12 hours apart from the previous dose, and wherein each does is administered with food; and

(b) said docetaxel is administered in an amount of about 50 to about 100 mg/m² (for example, 75 mg/m²) once in the treatment cycle.

Another embodiment of this invention is directed to a method of treating prostate cancer in a patient in need of such treatment, said treatment comprising administering an effective amount of lonafarnib in combination with an effective amount of docetaxel wherein:

(a) said lonafarnib is administered continuously for 1 to about 5 days of the treatment cycle in an amount of about 200 mg PO BID (i.e., orally twice per day) for each day of administration in the treatment cycle; and

(b) said docetaxel is administered in an amount of about 75 mg/m² once in the treatment cycle.

Another embodiment of this invention is directed to a method of treating prostate cancer in a patient in need of such treatment, said treatment comprising administering an effective amount of lonafarnib in combination with an effective amount of docetaxel wherein:

(a) said lonafarnib is administered continuously for 1 to about 5 days of the treatment cycle in an amount of about 200 mg PO BID (i.e., orally twice per day) for each day of administration in the treatment cycle, wherein each dose is administered about 12 hours apart from the previous dose; and

(b) said docetaxel is administered in an amount of about, 75 mg/m² once in the treatment cycle.

Another embodiment of this invention is directed to a method of treating prostate cancer in a patient in need of such treatment, said treatment comprising administering an effective amount of lonafarnib in combination with an effective amount of docetaxel wherein:

(a) said lonafarnib is administered continuously for 1 to about 5 days of the treatment cycle in an amount of about 200 mg PO BID (i.e., orally twice per day) for each day of administration in the treatment cycle, wherein each dose is administered about 12 hours apart from the previous dose, and wherein each does is administered with food; and

(b) said docetaxel is administered in an amount of about 75 mg/m² once in the treatment cycle.

Another embodiment of this invention is directed to any one of the methods of treating prostate cancer wherein said docetaxel is administered on the same day as said lonafarnib.

Another embodiment of this invention is directed to any one of the methods of treating prostate cancer wherein said lonafarnib is administered starting on day 1 of the cycle.

Another embodiment of this invention is directed to any one of the methods of treating prostate cancer wherein said docetaxel is administered on the first day of the treatment cycle.

Another embodiment of this invention is directed to any one of the methods of treating prostate cancer wherein said lonafarnib is started on the first day of the treatment cycle and said docetaxel is administered on the first day of the treatment cycle.

Another embodiment of this invention is directed to any one of the methods of treating prostate cancer where said lonafarnib is started on the first day of the treatment cycle and said docetaxel is started on the second day of the treatment cycle.

Another embodiment of this invention is directed to any one of the methods of treating prostate cancer where a treatment cycle is for 21 days.

Another embodiment of this invention is directed to any one of the methods of treating prostate cancer where the treatment cycle is repeated.

Another embodiment of this invention is directed to any one of the methods of treating prostate cancer wherein there are six treatment cycles.

Another embodiment of this invention is directed to any one of the methods of treating prostate cancer wherein said lonafarnib is continued as monotherapy after the last treatment cycle. In the monotherapy lonafarnib is administered in an amount of about 25 to about 200 mg PO BID. Usually, lonafarnib is administered at a dose of 200 mg PO BID. Usually each dose is administered 12 hours apart, and usually each dose is administered with food. The monotherapy with lonafarnib can be continued as long as the patient experiences stable disease, NED (no evidence of disease) or objective response (CR/PR), and manageable toxicity. Thus, for example, the lonafarnib monotherapy can be continued for 6 months after cessation of the combination therapy (i.e., after cessation of the therapy with docetaxel).

Lonafarnib (available from Schering-Plough Corporation as the Sarasar® brand of lonafarnib) is an FPT inhibitor having the formula:

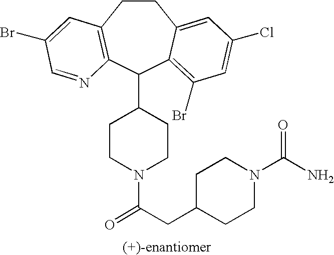

which compound can also be represented by the formula:

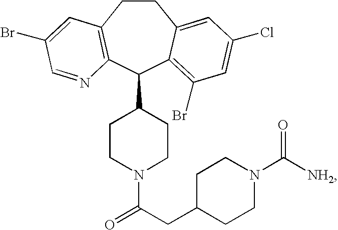

that is ((11R)4[2[4-(3,10-dibromo-8-chloro-6,11-dihydro-5H-benzo[5,6]cyclohepta-[1,2-b]pyridin-11yl)-1-piperazinyl]-2-oxoethyl]-1-piperidinecarboxamide)). This compound is described in U.S. Pat. No. 5,874,442 issued Feb. 23, 1999, and WO99/32118 published Jul. 1, 1999, the disclosures of which are incorporated herein by reference thereto.

The taxanes and the platinum coordinator complexes can be administered according to therapeutic protocols well known in the art for the treatment of a particular cancer (e.g., ovarian cancer or prostate cancer). Also, in accordance with the knowledge of the skilled clinician, the therapeutic protocols (e.g., dosage amounts and times of administration) can be varied in view of the observed effects of the administered therapeutic agents (e.g., lonafarnib, the taxane, and the platinum coordinator complex) on the patient, and in view of the observed responses of the ovarian cancer to the administered therapeutic agents.

The initial administration can be made according to established protocols known in the art, and then, based upon the observed effects, the dosage, modes of administration and times of administration can be modified by the skilled clinician.

Thus, in accordance with experience and knowledge, the practicing physician can modify each protocol for the administration of the therapeutic agents according to the individual patient's needs, as the treatment proceeds. All such modifications are within the scope of the present invention.

For example, the attending clinician, in judging whether treatment is effective at the dosage administered, will consider the general well-being of the patient as well as more definite signs such as relief of cancer-related symptoms (e.g., pain), inhibition of tumor growth, actual shrinkage of the tumor, or inhibition of metastasis. Size of the tumor, or tumor burden, can be measured by standard methods such as sequential measurements of serum CA-125 level or radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Response and progression to treatment can be evaluated according to criteria known in the art, such as the criteria proposed by the RECIST (Response Evaluation Criteria in Solid Tumors) committee (see, Therasse P, Arbuck S G, Eisenhauer E A et al., J Natl Cancer Inst 2000, 92:205-216, New guidelines to evaluate the response to treatment in solid tumors) with supplemental definitions of progression as published by Vergote et al., J Natl Cancer Inst 2000, 92:1534-1535, Re: new guidelines to evaluate the response to treatment in solid tumors [ovarian cancer]. Gynecological Cancer Intergroup.).

Progression-recurrence is defined according to the RECIST criteria and GCIG modifications and includes also: (a) occurrence (clinically or imaging signs) of any new lesion, (b) health status deterioration attributable to the disease, (c) death of any cause before progression is diagnosed, (d) CA 125 elevation as defined by the GCIG criteria, and (e) increase in measurable and/or non-measurable tumor as defined by the RECIST criteria.

Measurable disease can be defined as lesions that can accurately be measured in at least one dimension (longest diameter (LD) to be recorded) as ≧20 mm with conventional techniques (or as ≧10 mm with spiral CT scan). Nonmeasurable lesions can be defined as, all other lesions, including small lesions (LD<20 mm with conventional techniques or <10 mm with spiral CT scan) and truly nonmeasurable lesions. Lesions that are considered as truly nonmeasurable include: bone lesions, leptomeningeal disease, ascites, pleural/pericardial effusion, inflammatory breast disease, lymphangitis cutis/pulmonis, abdominal masses that are not confirmed and followed by imaging techniques, and cystic lesions. Measurable disease is the presence of at least one measurable lesion. If the measurable disease is restricted to a solitary lesion, its neoplastic nature should be confirmed by cytology/histology (techniques well known to those skilled in the art).

All measurable lesions up to a maximum of 5 lesions per organ and 10 lesions in total representative of all involved organs should be identifies as target lesions and recorded and measured at baseline. Target lesions should be selected on the basis of their size (lesion with the longest dimension) and their suitability for accurate repetitive measurements by one consistent method of assessment (either by imaging techniques or clinically). A sum of the longest dimension (LD) for all target lesions should be calculated and reported as the baseline sum LD. The baseline sum LD will be used as reference to further characterize the objective tumor response of the measurable dimension of the disease.

All other lesions (or sites of disease) should be identified as non-target lesions and should also be recorded at baseline. Measurements of these lesions are not required, but they should be followed as “absent”.

In the evaluation of target lesions: (1) a complete response (CR) means the disappearance of all target lesions; (2) a partial response (PR) means at least a 30% decrease in the sum of the longest diameter (LD) of target lesions, taking as reference the baseline sum LD; (3) stable disease (SD) means neither sufficient shrinkage to qualify for PR nor sufficient increase to qualify for PD (progressive disease), taking as reference the smallest sum LD since the treatment started; and (4) progressive disease (PD) means at least 20% increase in the sum of the LD of target lesions, taking as reference the smallest sum LD recorded since the treatment started or the appearance of one or more new lesions.

In the evaluation of non-target lesions: (1) a complete response (CR) means disappearance of all non-target lesions and normalization of tumor marker; (2) incomplete response/stable disease (SD) means persistence of one or more non-target lesions(s) and/or the maintenance of tumor marker level above the normal limits; and (3) progressive disease (PD) means appearance of one or more new lesions and/or unequivocal progression of existing non-target lesions.

The best overall response is the best response recorded from the start of the treatment until disease progression/recurrence (taking as reference for progressive disease the smallest measurements recorded since the treatment started). The patient's best response assignment will depend on the achievement of both measurement and confirmation criteria.

Other embodiments of this invention are directed to any one of the embodiments described above using about 25 to about 200 mg PO BID of lonafarnib (e.g., in the treatment cycle or in the monotherapy) wherein instead about 25 to about 350 mg PO BID (e.g., about 200 mg to about 350 mg PO BID, also for example, about 200 mg to about 250 mg, also for example 350 mg PO BID, also for example, 250 mg PO BID) is used. Other embodiments of this invention are directed to any one of the embodiments described above using about 200 mg PO BID of lonafarnib wherein instead about 200 to about 350 mg PO BID, or about 250 mg PO BID, or about 350 mg PO BID (in the treatment cycle or monotherapy) is used. Other embodiments include the administration of the doses higher than 200 mg PO BID in the treatment cycles for up to 7 days (e.g., 1 to about 7 days).

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

What is claimed is:

1. A method of treating taxane sensitive tumors in a patient in need of such treatment, said treatment comprising the discontinuous dosage administration of an effective amount of lonafarnib in combination with an effective amount of a taxane selected from the group consisting of: paclitaxel and docetaxel, and optionally, an effective amount a platinum coordinator complex selected from the group consisting of: carboplatin, cisplatin and oxaliplatin.

2. A method of treating ovarian cancer in a patient in need of such treatment, said treatment comprising administering lonafarnib in combination with paclitaxel and carboplatin, wherein:
(a) said lonafarnib is administered continuously for 1 to about 7 days of the treatment cycle in an amount of about 25 to about 200 mg PO BID (i.e., orally twice per day) for each day of administration in the treatment cycle;
(b) said paclitaxel is administered in an amount of about 135 mg/m² to about 185 mg/m² once during the treatment cycle; and
(c) said carboplatin is administered in an amount to provide an AUC (mg/ml×min) of about 4 to about 6 once during the treatment cycle.

3. The method of claim 2 wherein said lonafarnib is administered in an in an amount of about 100 mg twice per day on each day of administration.

4. The method of claim 3 wherein said paclitaxel is administered in an amount of about 175 mg/m² once during the treatment cycle.

5. The method of claim 4 wherein said carboplatin is administered in an amount to provide an AUC (mg/ml×min) of about 5 once during the treatment cycle.

6. The method of claim 5 wherein said paclitaxel and said carboplatin are administered once on day 1 of each treatment cycle.

7. The method of claim 2 wherein each dose of said lonafarnib is administered 12 hours apart.

8. The method of claim 7 wherein each dose of said lonafarnib is administered with food.

9. The method of claim 6 wherein each dose of said lonafarnib is administered 12 hours apart.

10. The method of claim 9 wherein each dose of said lonafarnib is administered with food.

11. The method of claim 2 wherein a treatment cycle is for 21 days.

12. The method of claim 6 wherein a treatment cycle is for 21 days.

13. The method of claim 9 wherein a treatment cycle is for 21 days.

14. The method of claim 10 wherein a treatment cycle is for 21 days.

15. The method of claim 2 wherein there is up to 6 treatment cycles.

16. The method of claim 11 wherein there is up to 6 treatment cycles.

17. The method of claim 12 wherein there is up to 6 treatment cycles.

18. The method of claim 13 wherein there is up to 6 treatment cycles.

19. The method of claim 14 wherein there is up to 6 treatment cycles.

20. The method of claim 2 wherein lonafarnib is continued as monotherapy in an amount of about 200 mg PO BID after the last treatment cycle.

21. The method of claim 15 wherein lonafarnib is continued as monotherapy in an amount of about 200 mg PO BID after the last treatment cycle.

22. The method of claim 16 wherein lonafarnib is continued as monotherapy in an amount of about 200 mg PO BID after the last treatment cycle.

23. The method of claim 17 wherein lonafarnib is continued as monotherapy in an amount of about 200 mg PO BID after the last treatment cycle.

24. The method of claim 18 wherein lonafarnib is continued as monotherapy in an amount of about 200 mg PO BID after the last treatment cycle.

25. The method of claim 19 wherein lonafarnib is continued as monotherapy in an amount of about 200 mg PO BID after the last treatment cycle.

26. A method of treating ovarian cancer in a patient in need of such treatment, said treatment comprising administering lonafarnib in combination with paclitaxel and carboplatin, wherein:
(a) said lonafarnib is administered continuously for 1 to about 5 days of the treatment cycle in an amount of about 25 to about 200 mg PO BID (i.e., orally twice per day) for each day of administration in the treatment cycle;
(b) said paclitaxel is administered in an amount of about 135 mg/m² to about 185 mg/m² once during the treatment cycle; and
(c) said carboplatin is administered in an amount to provide an AUC (mg/ml×min) of about 4 to about 6 once during the treatment cycle.

27. The method of claim 26 wherein said lonafarnib is administered in an in an amount of about 100 mg twice per day on each day of administration.

28. The method of claim 27 wherein said paclitaxel is administered in an amount of about 175 mg/m² once during the treatment cycle.

29. The method of claim 28 wherein said carboplatin is administered in an amount to provide an AUC (mg/ml×min) of about 5 once during the treatment cycle.

30. The method of claim 29 wherein said paclitaxel and said carboplatin are administered once on day 1 of each treatment cycle.

31. The method of claim 26 wherein each dose of said lonafarnib is administered 12 hours apart.

32. The method of claim 31 wherein each dose of said lonafarnib is administered with food.

33. The method of claim 30 wherein each dose of said lonafarnib is administered 12 hours apart.

34. The method of claim 33 wherein each dose of said lonafarnib is administered with food.

35. The method of claim 26 wherein a treatment cycle is for 21 days.

36. The method of claim 30 wherein a treatment cycle is for 21 days.

37. The method of claim 33 wherein a treatment cycle is for 21 days.

38. The method of claim 34 wherein a treatment cycle is for 21 days.

39. The method of claim 26 wherein there is up to 6 treatment cycles.

40. The method of claim 35 wherein there is up to 6 treatment cycles.

41. The method of claim 36 wherein there is up to 6 treatment cycles.

42. The method of claim 37 wherein there is up to 6 treatment cycles.

43. The method of claim 38 wherein there is up to 6 treatment cycles.

44. The method of claim 26 wherein lonafarnib is continued as monotherapy in an amount of about 200 mg PO BID after the last treatment cycle.

45. The method of claim 39 wherein lonafarnib is continued as monotherapy in an amount of about 200 mg PO BID after the last treatment cycle.

46. The method of claim 40 wherein lonafarnib is continued as monotherapy in an amount of about 200 mg PO BID after the last treatment cycle.

47. The method of claim 41 wherein lonafarnib is continued as monotherapy in an amount of about 200 mg PO BID after the last treatment cycle.

48. The method of claim 42 wherein lonafarnib is continued as monotherapy in an amount of about 200 mg PO BID after the last treatment cycle.

49. The method of claim 43 wherein lonafarnib is continued as monotherapy in an amount of about 200 mg PO BID after the last treatment cycle.

50. A method of treating ovarian cancer in a patient in need of such treatment, said treatment comprising administering lonafarnib in combination with paclitaxel and carboplatin, wherein:
(a) said lonafarnib is administered continuously for 1 to about 7 days of the treatment cycle in an amount of about 200 mg PO BID for each day of administration in the treatment cycle;
(b) said paclitaxel is administered in an amount of about 135 mg/m² to about 185 mg/m² once during the treatment cycle; and
(c) said carboplatin is administered in an amount to provide an AUC (mg/ml×min) of about 4 to about 6 once during the treatment cycle.

51. The method of claim 50 wherein said paclitaxel is administered in an amount of about 175 mg/m² once during the treatment cycle.

52. The method of claim 51 wherein said carboplatin is administered in an amount to provide an AUC (mg/ml×min) of about 5 once during the treatment cycle.

53. The method of claim 52 wherein said paclitaxel and said carboplatin are administered once on day 1 of each treatment cycle.

54. The method of claim 50 wherein each dose of said lonafarnib is administered 12 hours apart.

55. The method of claim 54 wherein each dose of said lonafarnib is administered with food.

56. The method of claim 53 wherein each dose of said lonafarnib is administered 12 hours apart.

57. The method of claim 56 wherein each dose of said lonafarnib is administered with food.

58. The method of claim 50 wherein a treatment cycle is for 21 days.

59. The method of claim 53 wherein a treatment cycle is for 21 days.

60. The method of claim 56 wherein a treatment cycle is for 21 days.

61. The method of claim 57 wherein a treatment cycle is for 21 days.

62. The method of claim 50 wherein there is up to 6 treatment cycles.

63. The method of claim 58 wherein there is up to 6 treatment cycles.

64. The method of claim 59 wherein there is up to 6 treatment cycles.

65. The method of claim 60 wherein there is up to 6 treatment cycles.

66. The method of claim 61 wherein there is up to 6 treatment cycles.

67. The method of claim 50 wherein lonafarnib is continued as monotherapy in an amount of about 200 mg PO BID after the last treatment cycle.

68. The method of claim 62 wherein lonafarnib is continued as monotherapy in an amount of about 200 mg PO BID after the last treatment cycle.

69. The method of claim 63 wherein lonafarnib is continued as monotherapy in an amount of about 200 mg PO BID after the last treatment cycle.

70. The method of claim 64 wherein lonafarnib is continued as monotherapy in an amount of about 200 mg PO BID after the last treatment cycle.

71. The method of claim 65 wherein lonafarnib is continued as monotherapy in an amount of about 200 mg PO BID after the last treatment cycle.

72. The method of claim 66 wherein lonafarnib is continued as monotherapy in an amount of about 200 mg PO BID after the last treatment cycle.

73. The method of claim 50 wherein said lonafarnib is administered for 1 to 5 days.

74. The method of claim 73 wherein said paclitaxel and said carboplatin are administered once on day 1 of each treatment cycle.

75. The method of claim 74 wherein the each dose of lonafarnib is administered 12 hours apart.

76. The method of claim 75 wherein a treatment cycle is 21 days and said treatment cycles are repeated.

77. The method of claim 76 wherein lonafarnib is continued as monotherapy at 200 mg PO BID after the last treatment cycle.

78. The method of claim 76 wherein said paclitaxel is administered in an amount of about 175 mg/m² once during the treatment cycle, and said carboplatin is administered in an amount to provide an AUC (mg/ml×min) of about 5 once during the treatment cycle.

79. The method of claim 78 wherein lonafarnib is continued as monotherapy at 200 mg PO BID after the last treatment cycle.

80. The method of claim 2 wherein said paclitaxel and said carboplatin are administered on day two of a treatment cycle.

81. The method of claim 26 wherein said paclitaxel and said carboplatin are administered on day two of a treatment cycle.

82. The method of claim 50 wherein said paclitaxel and said carboplatin are administered on day two of a treatment cycle.

83. The method of claim 73 wherein said paclitaxel and said carboplatin are administered on day two of a treatment cycle.

84. A method of treating prostate cancer in a patient in need of such treatment, said treatment comprising administering an effective amount of lonafarnib in combination with an effective amount of a taxane, wherein said lonafarnib is administered in a discontinuous dosing schedule.

85. A method of treating prostate cancer in a patient in need of such treatment, said treatment comprising administering an effective amount of lonafarnib in combination with an effective amount of docetaxel, wherein said lonafarnib is administered in a discontinuous dosing schedule.

86. A method of treating prostate cancer in a patient in need of such treatment, said treatment comprising administering an effective amount of lonafarnib in combination with an effective amount of docetaxel, wherein:
(a) said lonafarnib is administered continuously for 1 to about 7 days of the treatment cycle in an amount of about 25 to about 200 mg PO BID for each day of administration in the treatment cycle; and
(b) said docetaxel is administered in an amount of about 50 to about 100 mg/m² once in the treatment cycle.

87. The method of claim 86 wherein said lonafarnib is administered in an amount of about 200 mg PO BID.

88. The method of claim 86 wherein said docetaxel is administered in an amount of about 75 mg/m².

89. The method of claim 87 wherein said docetaxel is administered in an amount of about 75 mg/m².

90. The method of claim 86 wherein each dose of said lonafarnib is administered 12 hours apart.

91. The method of claim 89 wherein each dose of said lonafarnib is administered 12 hours apart.

92. The method of claim 90 wherein said lonafarnib is administered with food.

93. The method of claim 91 wherein said lonafarnib is administered with food.

94. The method of claim 86 wherein a treatment cycle is 21 days.

95. The method of claim 89 wherein a treatment cycle is 21 days.

96. The method of claim 90 wherein a treatment cycle is 21 days.

97. The method of claim 94 wherein said treatment cycle is repeated.

98. The method of claim 95 wherein said treatment cycle is repeated.

99. The method of claim 96 wherein said treatment cycle is repeated.

100. The method of claim 97 wherein said treatment cycle is repeated up to 6 times.

101. The method of claim 98 wherein said treatment cycle is repeated up to 6 times.

102. The method of claim 99 wherein said treatment cycle is repeated up to 6 times.

103. The method of claim 86 wherein said docetaxel is administered on a day when said lonafarnib is administered.

104. The method of claim 86 wherein said docetaxel is administered on the first day of a treatment cycle.

105. The method of claim 86 wherein said lonafarnib is started on day 1 of a treatment cycle.

106. The method of claim 86 wherein said lonafarnib is started on the first day of a treatment cycle and said docetaxel is given on the first day of a treatment cycle.

107. The method of claim 86 wherein docetaxel is given on day 2 of a treatment cycle.

108. The method of claim 107 wherein the lonafarnib is started on the first day of a treatment cycle.

109. A discontinuous dosing method of treating taxane sensitive tumors in a patient in need of such treatment, said treatment comprising administering an effective amount of lonafarnib in combination with the standard of therapy for said taxane sensitive tumors, said standard of therapy comprising the administration of the standard of care dose of said taxane.
